(19) United States
(12) Patent Application Publication
(10) Pub. No.: US 2023/0181556 A1
(43) Pub. Date: Jun. 15, 2023

(54) USE OF PSYCHOTROPIC DRUGS FOR THE TREATMENT OF CORONAVIRUS INFECTIONS

(71) Applicants: INSERM (INSTITUT NATIONAL DE LA SANTÉ ET DE LA RECHERCH MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ PARIS CITÉ, Paris (FR); UNIVERSITÉ PARIS-EST CRÉTEIL VAL DE MARNE, Creteil (FR)

(72) Inventors: Bruno VILLOUTREIX, Paris (FR); Philippe BEAUNE, Paris (FR); Marion LEBOYER, Cretéil CEDEX (FR); Ryad TAMOUZA, Créteil (FR); Rajagopal KRISHNAMOORTHY, Antony (FR); Vincent CALVEZ, Paris (FR); Anne-Geneviève MARCELIN, Paris (FR)

(21) Appl. No.: 17/999,298

(22) PCT Filed: May 18, 2021
(86) PCT No.: PCT/EP2021/063113

§ 371 Date: Nov. 18, 2022

(30) Foreign Application Priority Data
May 19, 2020 (EP) 20305517.3
Dec. 15, 2020 (EP) 20306570.1

(51) Int. Cl. A61K 31/4545; A61K 31/5415; A61K 31/496; A61K 31/40; A61K 31/495; A61K 31/343; A61K 31/5513; A61K 33/00; A61K 31/4045; A61K 31/465; A61K 31/554; A61K 31/135; A61K 31/19; A61K 31/4985; A61P 31/14; A61K 45/06

(52) U.S. Cl. CPC A61K 31/4545; A61K 31/5415; A61K 31/496; A61K 31/40; A61K 31/495; A61K 31/343; A61K 31/5513; A61K 33/00; A61K 31/4045; A61K 31/465; A61K 31/554; A61K 31/135; A61K 31/19; A61K 31/4985; A61P 31/14; A61K 45/06

(57) ABSTRACT

The coronavirus SARS-Cov-2 which started in Wuhan, China, in December 2019 induced a threat to global health. Patients with mental disorders were thought to be at increased risk of becoming infected for several reasons but very much to the inventors' surprise, these psychiatric units remained nearly empty during the lock-down period as if psychiatric patients were protected from SARS-Cov-2 infection. Thus the inventors mined the literature to identify approved drugs with in vitro antiviral activities with a special emphasis on psychotropic drugs and compared these molecules using chemoinformatics strategies to the drugs most commonly used in their psychiatric department. A large number of compounds were found to be cationic amphiphilic drugs (CADs) and as such tend to also be phospholipidosis inducers in vitro (PLD). Several molecules most commonly used in their department were also found to have in vitro antiviral activity and/or can be clustered with compounds with known in vitro antiviral activity. Thus, drugs most commonly used for mental disorders with known in vitro antiviral activities are often CADs. In silico and in vitro analyses of these compounds together with the observations made in the department strongly suggest that commonly used psychotropic drugs and some related anti-histamine agents used as anxiolytics should protect psychiatric patients from SARS-Cov-2 infection.

### FIELD OF THE INVENTION

The present invention is in the field of medicine and in particular virology.

### BACKGROUND OF THE INVENTION

The coronavirus SARS-Cov-2 which started in Wuhan, China, in December 2019 induced a threat to global health. In Mar. 11, 2020, the WHO declared COVID-19 as a pandemic. As of yet, as it seems to spread very actively, it has infected more than 185 countries with more than 4, 100, 000 confirmed cases, and more than 280,000 deaths as of May 10, 2020. This pandemic follows several highly pathogenic human coronaviruses infections including SARS-CoV in 2002 with a death rate of 10% and MERS-CoV in 2012 with a death rate of 36%.

To date, no antiviral drugs have been approved neither for the treatment, nor for the prevention of the SARS-CoV, SARS-CoV2 or MERS-CoV infections. As of May 12, 2020, 1,429 clinical trials are ongoing (https://clinicaltrials.gov) and several in vitro drug repurposing studies are being performed. Since the number of cases continue to grow worldwide and as drug development is extremely time-consuming and costly, there is an urgent need to find a rationale for the repurposing of clinically approved compounds which could be validated in epidemiological settings in vitro and then in clinical trials. Considering that the pharmaco-dynamics, -kinetics and toxicology of these compounds are already established, the use of clinically approved drugs with few side effects would be invaluable in terms of bypassing costs and time associated with drug development.^{1, 2} In particular, we believe that there is great potential in exploring patients with mental disorders as this could lead to the identification of specific drugs to be used for prevention.

It became clear, in the past few weeks, that a particular relationship exists between SARS-CoV-2 infection and psychiatric disorders. Patients with mental disorders were thought to be at increased risk of becoming infected for several reasons; cognitive impairment possibly diminished efforts to follow protection rules with little awareness of risk, timely access to health services may be delayed because of discrimination associated with mental illnesses, confined conditions in psychiatric wards may speed up the spread of infections, as well as physical co-morbidities (diabetes, cardio-vascular disorder, obesity), which put these patients at increased risk of severe forms of Covid-19 infections.³ For all these reasons in France and elsewhere in the world, psychiatric departments were rapidly emptied to create dedicated units for patients with psychiatric disorders and Covid19.⁴ Very much to our surprise, these units remained nearly empty during the lock-down period as only a small number of psychiatric patients were reported to have comorbid Covid-19, as if psychiatric patients were protected and thus at reduced risk of infection by SARS-CoV-2.

In particular, it has been repeatedly reported that treatments used for psychiatric disorders, either isolated or in combination, had either anti-infectious or anti-inflammatory properties in vitro. For example, Lithium chloride (LiCl), the well-known mood stabilizer has antiviral effects as it can inhibit the infection of some RNA and DNA viruses.⁵ It has also been described that several phenothiazines synergize with antibiotics and possess their own intrinsic antibacterial activity, although their use as antibiotic adjuvant is limited by their cytotoxicity at the required dosage.⁶ Nicotine is also a drug largely prescribed to psychiatric patients (#1 in our hospital department). As such, it is quite interesting to note that smokers seem to be protected from SARS-CoV-2 infection. The mechanisms are still unclear but quite likely involve the binding of nicotine to the nAcetyl Choline Receptor (nAChR) expressed in many tissues including lung and brain. Of note, neurologic symptoms or pathologies are quite frequent in Covid-19 patients. Possibly, Nicotine could block the entry of the virus in the brain as an AChR blocker, and/or downregulate ACE2 receptors. In addition, Nicotine could also act through its anti-inflammatory effects.

### SUMMARY OF THE INVENTION

As defined by the claims, the present invention relates to use of psychotropic and structurally related antihistaminic drugs for the treatment of coronavirus infections.

### DETAILED DESCRIPTION OF THE INVENTION

The coronavirus SARS-Cov-2 which started in Wuhan, China, in December 2019 induced a threat to global health. For the time being, no treatments or vaccines are available while the number of cases continues to grow. Patients with mental disorders were thought to be at increased risk of becoming infected for several reasons but very much to the inventors' surprise, these psychiatric units remained nearly empty during the lock-down period as if psychiatric patients were protected from SARS-Cov-2 infection. Thus the inventors mined the literature to identify approved drugs with in vitro antiviral activities with a special emphasis on psychotropic and related antihistaminic drugs and compared these molecules using chemoinformatics strategies to the drugs most commonly used in their psychiatric department (i.e., several antihistaminic drugs are commonly prescribed to psychiatric patients). A large number of compounds were found to be cationic amphiphilic drugs (CADs) and as such tend to also be phospholipidosis inducers in vitro (PLD) and some can be also considered as lysosomotropic agents. Several molecules most commonly used in their medical department were also found to have in vitro antiviral activity and/or can be clustered with compounds with known in vitro antiviral activity. Thus, drugs most commonly used for mental disorders with known in vitro antiviral activities are often CADs. Such compounds tend to interfere with virus entry and also impede intracellular trafficking. Analysis of these compounds together with the observations made in the department strongly suggest that commonly used psychotropic drugs and some related anti-histamine agents used as anxiolytics should protect psychiatric patients from SARS-Cov-2 infection.

Accordingly, the first object of the present invention relates to a method of treating a coronavirus infection in a subject in need thereof comprising administering to the patient a therapeutically effective amount of one drug selected from the group consisting of Desloratadine, Promethazine, Loratadine, Azelastine, Pizotifen, Bromodiphenhydramine, Diphenhydramine, Cyproheptadine, Oxomemazine, Cetirizine, Hydroxyzine, Alimemazine, Amisulpride, Aripiprazole, Citalopram, Clozapine, Cyamemazine, Diazepam, Escitalopram, Lorazepam, Melatonin, Quetiapine, Sertraline, Valproate, Zopiclone, Rupatadine, Azatadine, Promazine, Profenamine, Methdilazine, Perazine, Perphenazine, Ketotifen, Orphenadrine and Lithium.

In particular, the drug is selected from the group consisting of Desloratadine, Promethazine, Loratadine and Azelastine. More particularly, the drug is selected from the group consisting of Desloratadine, Promethazine and Loratadine. Even more particularly, the drug is selected from the group consisting of Desloratadine and Loratadine. Even more particularly, the drug is Desloratadine.

As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. These coronaviruses infect a variety of mammals and birds. They cause respiratory infections (common), enteric infections (mostly in infants>12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), and SARS-coronavirus (Center for Disease Control), in particular SARS-Cov1 and SARS-Cov2.

In particular, the method of the present invention is suitable for the treatment of Severe Acute Respiratory Syndrome (SARS). More particularly, the method of the present invention is suitable for the treatment of COVID-19.

In some embodiments, the subject can be human or any other animal (e.g., birds and mammals) susceptible to coronavirus infection (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant. In some embodiment, the subject is immunocompromised. An immunocompromised subject is a subject suffering from immunodeficiency due to an impaired immune system. This impairment can be due to therapeutic treatment. In some embodiment, the subject cannot receive vaccines or refuses vaccines. In some embodiment, the subject is undergoing organ transplant.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The drug according to the present invention is selected among cationic amphiphilic drugs (CAD) and/or in vitro phospholipid inducer agents (PLD) and/or molecules structurally similar to psychotropic drugs.

As used herein, the term “cationic amphiphilic drug” refers to a compound characterized by hydrophobic-aromatic ring systems and a side chain that carries one (or more) ionizable amine functional group. In particular, cationic compounds are molecules with an amine group with a basic pKa value>7.1, which indicates that these compounds are mainly protonated a low pH values (i.e. in the endosomal-lysosomal system, pH values are in the range of 6.5 to about 4.5). Amphiphilic compounds have both hydrophilic and hydrophobic chemical moieties in their structures and this global property can be estimated by log P values (i.e., an estimation of the lipophilic character of small drug-like compounds).

In some embodiments, the cationic amphiphilic drug induces phospholipidosis as described in the EXAMPLE 1 and as characterized by Muehlbacher M, Tripal P, Roas F, Kornhuber J. Identification of drugs inducing phospholipidosis by novel in vitro data. Chem Med Chem 2012; 7(11): 1925-34.

In some embodiments, at least two drugs of the present invention are administered to the subject.

In some embodiment, the drug of the present invention is administered to the subject in combination with at least one other therapeutic agent, preferably in combination with at least one other antiviral agent, more preferably in combination with at least one other antiviral agent selected from the group consisting of remdesivir, lopinavir, and ritonavir. In some embodiment, the drug of the present invention is administered to the subject in combination with Azithromycin. In some embodiments, the drug of the present invention is administered to the subject in combination with at least one drug selected from the group consisting of Chloroquine, Hydroxyl-chloroquine, Nicotine, Clemastine, Cloperastine, and Haloperidol.

In some embodiment, the drug of the present invention is administered with at least one other therapeutic agent, preferably in combination with at least one antibody, more particularly an anti-spike antibody. As example, antibodies may be bamlanivimab, casirivimab, cilgavimab, regdanvimab, sotrovimab, tixagevimab, etesevimab, imdevimab, tocilizumab, COR101, MR17, SR4, COV2-2064, VHH-72, TY-027 or JMB2002 used alone or combined.

In some embodiments, the drug of the present invention is administered with at least one other therapeutic agent, preferably in combination with at least one vaccine. As example, vaccines may be mRNA-1273, BNT162b2, AZD1222, CVnCoV vaccine, NVX-CoV2373, BBIBP-CorV, Ad26.COV2.S, Ad5-nCov, Gam-COVID-Vac, CoVLP, BBV152-COVAXIN, AG0302-COVID19, SCB-2019, QazCovid-in, UB-612 or EpiVacCorona, used alone or combined.

In some embodiments, the present invention relates to i) the drug of the present invention, and ii) at least one other therapeutic agent, as a combined preparation for simultaneous, separate or sequential use in the treatment of a coronavirus infection.

As used herein, the term “simultaneous use” denotes the use of the drug according to the invention and at least one other therapeutic agent occurring at the same time.

As used herein, the term “separate use” denotes the use of a drug according to the invention and at least one other therapeutic agent not occurring at the same time.

As used herein, the term “sequential use” denotes the use of a drug according to the invention and at least one other therapeutic agent occurring by following an order.

According to the invention, the cationic amphiphilic drug is administered to the patient in a therapeutically effective amount. By a “therapeutically effective amount” is meant a sufficient amount of the active ingredient for treating or reducing the symptoms of the coronavirus infection at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the cationic amphiphilic drug is combined with pharmaceutically acceptable excipients, and optionally sustained-release matrices, such as biodegradable polymers, to form pharmaceutical compositions. The term “Pharmaceutical” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1: Cytopathic effect (i.e. structural changes in a host cell resulting from a viral infection) measured as a function of concentrations of Desloratadine (A), Azelastine (B) or Promethazine (C).

### EXAMPLE 1

Methods

To explore psychotropic drugs and rationalize putative molecular mechanisms, we first identified the most commonly used drugs in hospital settings and treatments massively used in private practice (i.e., consumption of all the drugs in the Psychiatric department of Henri Mondor Hospital, Creteil, France). We ended up with 18 drugs reported in Table 1. Moreover, we selected ten other drugs used in different therapeutic areas with known in vitro antiviral activities through literature mining (see also recent reviews).^{9, 10} In these previous studies, in vitro drug repurposing strategies have been carried out on viruses such as HIV, MERS, SARS, and/or Ebola among others.^{11, 12} Yet one recently reported repurposing studies has been performed on SARS-CoV-2.¹³ These ten molecules are presented in the bottom part of Table 1. Chemoinformatics strategies were applied to compare these ten drugs with known antiviral activities with the 18 molecules most commonly used in our department. These involved calculation of molecular descriptors (pKa, log P, fingerprints) and then clustering compounds by chemical similarity.

We observed that all ten reference compounds (e.g., chlorpromazine, promethazine, chloroquine, clomifene) with in vitro antiviral activities belong to a wide group of chemicals commonly referred to as cationic amphiphilic drugs or CADs. These molecules are characterized by hydrophobic-aromatic ring systems and a side chain that carries one (or more) ionizable amine functional group. To define the CAD of the selected compounds, we used computed pKa and log P values as reported in.¹⁴ We considered cationic compounds as molecules with an amine group (the most basic group was selected) with a basic pKa value>7.1, which indicates that these substances are mainly protonated a low pH values (i.e. in the endosomal-lysosomal system, pH values are in the range of 6.5 to about 4.5). Computation of pKa values were carried out with the ChemAxon chemistry toolkit (https://chemaxon.com/).

Amphiphilic compounds have both hydrophilic and hydrophobic chemical moieties in their structures and this global property can be estimated by log P values (i.e., an estimation of the lipophilic character of small drug-like compounds). DataWarrior was used to compute log P values.¹⁵ CADs often induce phospholipidosis (drug-induced phospholipidosis or PLD) in vitro.¹⁶ Hundreds of drugs have been tested in vitro to assess if they induce phospholipidosis.^{14, 17} When experimental data are not available, the property can also be predicted.^{17, 18}

Compounds were also grouped into families using similarity measures computed via the statistical package R and the ChemMine chemical toolkit and the implemented hierarchical clustering method was used.¹⁹ Similarity analyses were also performed with the 2D-Rubber Band Scaling approach applied to different similarity criterion.¹⁵

Chemoinformatics clustering analysis was also carried out over a collection of 4642 approved drugs. The clustering was performed by combining structural similarities found by the MACCS fingerprints or the count for the presence of specific organic functions or the consensus between MACCS fingerprints and the organic function count. A total of 3040 clusters were first identified. Then, focusing in the appropriate areas of the chemical space (i.e., compounds predicted to be CAD) and similar with the drugs mentioned in Table 1 allowed for the selection of 17 other molecules (Table 2).

Results:

Ten reference drugs (anti-malarial and anti-histamine agents and psychotropic drugs) with known antiviral activities were selected (Table 1). Independently of their exact chemical structures (not shown), the first observation is that they are all CADs. They are also known or predicted PLD compounds. For the 18 most commonly prescribed psychotropic drugs (Table 1), we note that, if we set aside lithium which is of different nature as not an organic molecule, among the remaining 17 molecules, 14 are also CADs and/or PLD.

There are different possibilities to group compounds in families. We clustered compounds by comparing chemical substructures and the presence of functional chemical groups. Two clusters with five or more members were identified. Interestingly, this approach groups compounds with known in vitro antiviral activities such as Chlorphenoxamine with commonly prescribed drugs such as Escitalopram. Similarly, Promethazine that is known to have in vitro antiviral activities (Table 1) is clustered with commonly prescribed drugs such as Cyamemazine or Alimemazine while the more structural diverse and commonly prescribed Aripiprazole compound can also be considered to belong to this family of molecules. Antimalarial compounds, Chloroquine and Hydroxychloroquine, form a small cluster but are also closely related to Clomifene, a molecule used to treat fertility disorders (i.e., all these compounds are neighbors in the 2D space).

The other approved drugs that cluster with the compounds most commonly given to psychiatric patients are reported Table 2. These molecules have therefore the potential to act on Covid-19 according to our chemoinformatics analysis.

Discussion:

Drugs against SARS-CoV-2 could operate at different stages of the virus lifecycle. To protect the population, acting on the virus entry phase through drug repurposing is an attractive solution. Different strategies can be envisioned, from specific inhibition of some proteases and receptors, to more fundamental mechanisms involving endocytosis or both. We present below our analysis on psychotropic drugs and some related molecules and suggest that many of these drugs impede virus entry and/or intracellular trafficking.

While not fully understood, the SARS-CoV-2 lifecycle comprises several steps: cell attachment and cell entry and intracellular trafficking, viral uncoating, nucleotide replication and viral assembly, and final release of viral genetic material in the cytoplasm.⁸ A critical event for entering the cells involves the binding of the SARS-CoV-2 spike glycoprotein RBD domain to the angiotensin converting enzyme 2 (ACE2) receptor.²⁰ Subsequently the virus enters the endosomes and eventually fuse viral and lysosomal membranes. To fuse membranes, the spike protein needs to be proteolytically activated (i.e., cleaved with associated conformational changes) by the transmembrane surface serine protease TMPRSS2 or other proteases and endosomal/lysosomal cathepsins while furin preactivation assists SARS-CoV-2 entry in some types of cells.^{8, 20-22} The SARS-CoV-2 virus entry and intracellular trafficking therefore appear to involve the endosomal (clathrin-dependent or not)/lysosomal pathway and complex autophagy processes suggesting that targeting endocytosis could be of potential therapeutic interest.^{23, 24}

The ten selected drugs used as reference compounds with known in vitro antiviral activities are reported in Table 1. These compounds are CADs, characterized by hydrophobic-aromatic ring systems and an overall hydrophobic side chain that carries one (or more) ionizable amine functional group.⁹ CADs can be partially charged at physiological pH but are protonated in the more acidic endolysosomal compartments. When protonated, the drugs are trapped in lysosomes and can severely perturb cell functions and trafficking. Related with these observations, it is known that many CADs induce phospholipidosis (drug-induced phospholipidosis or PLD), a situation characterized by the accumulation of phospholipids within the lysosome resulting from several mechanisms such as direct binding of the drugs to the membranes and/or inhibition of enzymes, raise of the pH value of the endolysosomal compartments toward neutrality . . . ).¹⁶ Among the ten reference compounds, eight are also psychotropic drugs and/or anti-histamine agents that have known in vitro antiviral activity (Table 1). According to our calculation, these molecules are all CADs. They are also known or predicted PLD compounds. Further, it we take the example of a compound from another therapeutic class such as Chloroquine, this anti-malarial compound is a CAD and a PLD compound. It is a lysosomotropic agent that accumulates in acidic organelles such as endosomes and lysosomes and neutralizes their pH thereby inhibiting protease activities with subsequent altered cleavages of the spike proteins damaging some events required for virus entry and possibly affecting the function of ACE2 (not shown)²⁵. Another molecule like Chlorpromazine is a CAD and PLD agent and is known to inhibit clathrin-medicated endocytosis (not shown).²⁶ If we compare the chemical structure (global structure or the presence of chemical substructures) and physico-chemical properties (Table 1) of these ten reference compounds with known in vitro antiviral activity with the 18 most commonly prescribed psychiatric medications (Table 1), we note that among these, if we set aside lithium, 14 are CADs and/or PLD and several of these molecules can be clustered and/or share many common chemical substructures (i.e., they are chemically related and many should follow the well-established similarity property principle which states that similar compounds have similar biological properties). In this context, it seems reasonable to speculate that many commonly used psychiatric drugs protect patients from SARS-CoV-2 via perturbation of the endo-lysosomal pathway with potential impairments of autophagy processes.

In parallel, we also searched for evidences that these 18 most commonly used psychotropic drugs in our department could have known in vitro antiviral activities, even if the mechanisms are not known and even if not specifically documented for SARS-CoV-2. Among these 18 molecules, at least ten have documented antiviral activities (Table 1). For some viruses such as Ebola, it has even been shown that drug combination could be beneficial.²⁷ The combination of for instance Toremifene (a CAD drug and predicted PLD by comparison with the highly similar compound tamoxifen, a known PLD agent) used in the treatment of advanced breast cancer with the anti-depressant Sertraline molecule (a CAD agent and PLD compound) can be used as an example. We propose that combining psychotropic drugs that have low adverse drug reaction properties with anti-histamine drugs and/or Nicotine could be of interest for the prevention of SARS-CoV-2 infection. Of importance, Tomerifen was initially proposed to possess anti-Ebola activities through destabilization of the Ebola GP glycoprotein, essential for virus entry into the cells (i.e., like the spike protein of SARS-CoV-2), but more recent investigations suggest that this drug acts because it is a CAD compound that impairs various endolysosomal functions and proteins.²⁸

Our analysis suggests that psychotropic drugs (including some anti-histamine agents used as anxiolytics) could, in association or not with nicotine, protect the population from SARS-CoV-2 during the virus entry phase and intracellular trafficking essentially by interfering with the endolysosomal pathway and further interactions with some specific receptors. Chemically, these compounds tend to all be cationic amphiphilic drugs and/or in vitro phospholipidosis inducers. Further experimental assessments need to be performed but chemical and clinical evidences strongly suggest that a preventive treatment is at hand by repurposing the reported psychotropic drugs against SARS-CoV-2 infection.

Starting from the two lists of molecules found by clinical observations and via chemoinformatics strategies (Table 1 and Table 2), we then text-mined drug repository databases in search for secondary effects, doses commonly prescribed and the mode of administration. Second, we further investigated the chemistry of the compounds via computational analysis looking for the presence of toxicophores (i.e., chemical structures that can lead to adverse drug reactions in some population). Based upon this analysis, we propose a top priority list of molecules that should have very limited secondary effects in the general population for clinical trials consisting of Desloratadine, Promethazine, Azelastine and Loratadine.

The second priority list of molecules include: Pizotifen, Bromodiphenhydramine, Diphenhydramine, Cyproheptadine, Oxomemazine, Cetirizine, and Hydroxyzine.

The third priority list of drugs involve: Alimemazine, Amisulpride, Aripiprazole, Citalopram, Clozapine, Cyamemazine, Diazepam, Escitalopram, Lorazepam, Melatonin, Quetiapine, Sertraline, Valproate, Zopiclone, Rupatadine, Azatadine, Promazine, Profenamine, Methdilazine, Perazine, Perphenazine, Ketotifen and Orphenadrine.

As mentioned above, these molecules could be combined or administrated with, for instance, Nicotine or antiviral agents or antibodies or vaccines.

To illustrate the efficiency of our invention, we tested in vitro three molecules from our top priority list (Loratadine was not tested as it needs to be metabolized, in the human body, to its main active form, Desloratadine).

### EXAMPLE 2

Virus Strain Used for the Assays

SARS-CoV-2 clinical isolates D614G (GenBank accession number MW322968) was isolated from a SARS-CoV-2 RT-PCR confirmed patients by inoculating Vero cells with sputum sample or nasopharyngeal swabs in our biosafety level-3 (BSL-3) facility. Viral stock were generated using one passage of isolate on Vero cells. Titration of viral stock was performed on Vero E6 by the limiting dilution assay allowing calculation of tissue culture infective dose 50% (TCID50).

Evaluation of Antiviral Activities of the Drugs

One day prior to the assay, Vero-E6 cells (ATCC® CRL-1586) were plated in a 96-wells flat bottom tissue culture treated plate at a density of 2×10⁴ cells/well. On the day of the assay, Vero-E6 cells were pre-treated with the different concentration (i.e. 10 μM, 5 μM and 2 μM) of the indicated drugs diluted in Dulbecco's Modified Eagles Medium (DMEM). After 1 h, the cells were infected with 50 μl of a viral inoculum at 1×10³ TCID50/ml and incubated for 2 h at 37° C. to allow infection. Then, the virus-drug mixture was removed, cells were rinsed 3 times with PBS and were further cultured with fresh drug-containing medium (DMEM 5% FBS) for 3 days until microscopic examination was performed. Cytotoxicity of the tested drugs were determined in the same experiment in mock infected cells. The half maximal inhibitory concentration (IC₅₀) were analyzed by non-linear regression using a four-parameter dosage-response variable slope model with the GraphPad Prism 8.0.2 software (GraphPad Software, USA).

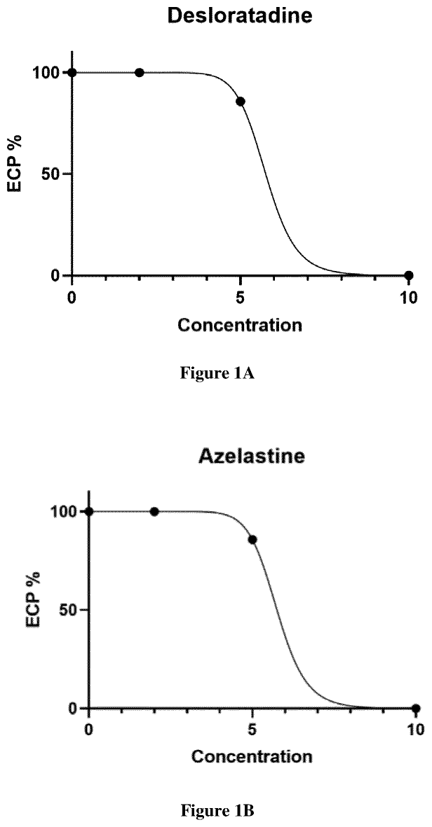

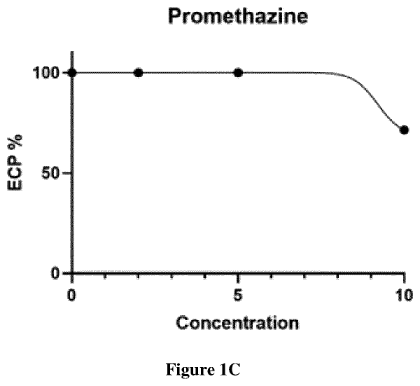

The results are shown in FIG. 1 and Table 3, highlighting the antiviral activity of Desloratadine (FIG. 1A), Azelastine (FIG. 1B) and Promethazine (FIG. 1C). These results demonstrate the efficiency of psychotropic and structurally related antihistaminic drugs for the treatment of coronavirus infections. These molecules were not found to be cytotoxic.

Conclusion:

These results are concomitants with other experiments. In Hou et al. (Chemico-Biological Interactions 338 (2021) 109420), the antiviral effect of Loratadine and Desloratadine was tested. These two molecules were efficient to block spike protein-ACE2 interaction, thus inhibiting SARS-CoV-2 entry to cells. No cytotoxicity was observed under 20 μM concentrations.

Here, we demonstrated the antiviral effects of psychotropic and structurally related antihistaminic drugs against SARS-CoV-2 infection and propose some already approved drugs that could be repositioned for the treatment against COVID-19.

Tables:

TABLE 1
Main psychotropic drugs and reference compounds used to develop our rationale.

| | | | | | | |
|---|---|---|---|---|---|---|
| | | | Number of | | | |
| Name | | | basic N/pKa | | PLD | Examples of |
| (rank of use in | | | of the most | | (experimental | published in vitro |
| our hospital) | Class | cLogP | basic group | CAD | or predicted) | antiviral activity |

18 main psychotropic drugs used in our department

| | | | | | | |
|---|---|---|---|---|---|---|
| Alimemazine (15) | anxiolytic | 4.2 | 1/9.42 | y | y (predicted) | not known |
| Amisulpride (>15) | anti- | 0.9 | 1/7.05 | n | y | not known |
| | psychotic | | | | | |
| Aripiprazole (8) | anti- | 4.4 | 1/7.46 | y | y | Drug combination |
| | psychotic | | | | | for Ebola virus |
| | | | | | | (J Infect Dis 2018; |
| | | | | | | 218(suppl_5): |
| | | | | | | S672-S8) |
| Cetirizine (15) | anti- | 2.1 | 1/7.42 | y | n (predicted) | HRV (Antiviral |
| | histamine | | | | | Res 2009; 81(3): |
| | anxiolytic | | | | | 226-33.; PLoS |
| | | | | | | One 2017; 12(7): |
| | | | | | | e0165415) |
| Citalopram (>15) | anti- | 2.8 | 1/9.78 | y | y | HIV |
| | depressant | | | | | (J Neuroimmune |
| | | | | | | Pharmacol 2007; |
| | | | | | | 2(1): 120-7) |
| Clozapine (2) | anti- | 3.2 | 1/7.35 | y | y | Inhibition of |
| | psychotic | | | | | Epstein-Barr |
| | | | | | | Virus Lytic |
| | | | | | | Reactivation HIV |
| | | | | | | (Viruses 2019; |
| | | | | | | 11(5); Schizophr |
| | | | | | | Res 1997; 25(1): |
| | | | | | | 63-70.) |
| Cyamemazine (10) | anti- | 4.0 | 1/9.42 | y | y (predicted) | not known |
| | psychotic | | | | | |
| Diazepam (7) | anti- | 2.9 | 0/2.92 | n | n | not known |
| | depressant | | | | | |
| Escitalopram (>15) | anti- | 2.7 | 1/9.78 | y | y (predicted) | not known |
| | depressant | | | | | |
| Hydroxyzine (>15) | anti- | 3.0 | 1/7.77 | y | y | Selective inhibition |
| | histamine | | | | | of hepatitis C virus |
| | anti- | | | | | infection (Antimicrob |
| | depressant | | | | | Agents Chemother 2014; |
| | | | | | | 58(6): 3451-60) |
| Lithium (4) | mood- | NA | 0/−4.2 | n | n | Antiviral effect of |
| | stabilizing | | | | | lithium chloride on |
| | | | | | | mammalian |
| | | | | | | orthoreoviruses |
| | | | | | | (Microb Pathog |
| | | | | | | 2016; 93: 152-7) |
| Lorazepam (3) | anxiolytic | 2.9 | 0/−2.2 | n | n | not known |
| Melatonin (9) | anti- | 1.5 | 0 | n | y | Possible roles |
| | depressant | | | | | in bacterial and |
| | | | | | | viral infections |
| | | | | | | (Recent Pat Endocr |
| | | | | | | Metab Immune |
| | | | | | | Drug Discov 2012; |
| | | | | | | 6(1): 30-9) |
| Nicotine (1) | | 1.2 | 1/8.6 | partial | y (predicted) | Inhibits the |
| | | | | | | production of pro- |
| | | | | | | inflammatory |
| | | | | | | cytokines in mice |
| | | | | | | infected with cox- |
| | | | | | | sackievirus B3 |
| | | | | | | (Life Sci 2016; |
| | | | | | | 148: 9-16) |
| Quetiapine(11) | anti- | 2.7 | 1/7.06 | y | y | not known |
| | psychotic | | | | | |
| Sertraline (>15) | anti- | 4.2 | 1/9.85 | y | y | Ebola virus |
| | depressant | | | | | Zika Virus |
| | | | | | | HIV in vivo |
| | | | | | | (J Neuroimmune |
| | | | | | | Pharmacol 2007; |
| | | | | | | 2(1): 120-7; Sci |
| | | | | | | Transl Med 2015; |
| | | | | | | 7(290): 290ra89; |
| | | | | | | Cell Host Microbe |
| | | | | | | 2016; 20(2): 259-70) |
| Valproate (5) | anti- | 2.2 | 0/NA | n | n | HSV (Virus Res |
| | depressant | | | | | 2016; 214: 71-9) |
| Zopiclone (6) | sedative | 0.71 | 1/6.89 | n | y (predicted) | not known |

10 reference compounds with known in vitro antiviral activity used for comparison with the above molecules

| | | | | | | |
|---|---|---|---|---|---|---|
| Chloroquine (NA) | anti- | 4.0 | 2/10.32 | y | y | MERS, SARS, |
| | malarial | | | | | Filovirus |
| | | | | | | (Sci Transl Med |
| | | | | | | 2015; 7(290): |
| | | | | | | 290ra89) |
| Chlorphenoxamine | anti- | 3.4 | 1/8.87 | y | y (predicted) | MERS, SARS |
| (>15) | histamine | | | | | (Antimicrob Agents |
| | | | | | | Chemother 2014; |
| | | | | | | 58(8): 4885-93) |
| Chlorpromazine | anti- | 4.6 | 1/9.2 | y | y | MERS, SARS |
| (>15) | psychotic | | | | | (Antimicrob Agents |
| | anti | | | | | Chemother 2014; |
| | histamine | | | | | 58(8): 4885-93) |
| Clemastine (>15) | anti- | 4.6 | 1/9.55 | y | y | SARS-CoV-2 |
| | histamine | | | | | (Nature. 2020 July; |
| | | | | | | 583(7816): 459-468) |
| Clomifene (NA) | women | 5.1 | 1/9.31 | y | y | Ebola, HCV |
| | infertility | | | | | (Microbes Infect |
| | | | | | | 2013; 15(1): 45-55; |
| | | | | | | Sci Transl Med 2013; |
| | | | | | | 5(190): 190ra79) |
| Clomipramine (>15) | anti- | 4.5 | 1/9.2 | y | y | MERS, SARS |
| | depressant | | | | | (Antimicrob Agents |
| | | | | | | Chemother 2014; |
| | | | | | | 58(8): 4885-93) |
| Cloperastine (>15) | anti- | 4.6 | 1/8.82 | y | y | SARS-CoV-2 |
| | histamine | | | | | (Nature. 2020 July; |
| | | | | | | 583(7816): 459-468) |
| Haloperidol (>15) | anti- | 4.3 | 1/8.05 | y | y | SARS-CoV-2 |
| | psychotic | | | | | (Nature. 2020 July; |
| | | | | | | 583(7816): 459-468) |
| Hydroxyl- | anti- | 3.1 | 2/9.76 | y | y | SARS-Cov-2 |
| chloroquine (NA) | malarial | | | | | (Nature. 2020 July; |
| | | | | | | 583(7816): 459-468) |
| Promethazine (>15) | anti- | 3.9 | 1/9.05 | y | y | Ebola (Sci Transl Med |
| | histamine | | | | | 2015; 7(290): 290ra89) |

The first part of the table lists the main 18 drugs used in our department and/or in private practice. The second part represents a list of ten molecules with known in vitro antiviral activity that were used for comparison with our compounds. It turned out that several of these ten reference molecules are also used in our hospital setting and/or in private practice. Further, mining of the literature indicated that several molecules commonly used in our department also have in vitro antiviral activities. This information was thus added to the table.

We searched for some common properties that could be shared by these compounds and more specifically, we initially tried to answer two questions: are these molecules cationic amphiphilic drugs (CADs)? and could they be inducers of phospholipidosis, at least in vitro (PLD)?. The scientific rationale is that several CADs have in vitro antiviral activity (e.g., Chloroquine, Hydroxychloroquine, Chlorpromazine, Promethazine, Sertraline or Clomifen) and that CADs tend to also be PLD.^{16, 19} This type of molecules could possibly impede virus entry into the host cells due to the intrinsic properties of CADs. These molecules should also impair the endolysosomal pathway and intracellular trafficking and can bind or regulate some specific receptors and enzymes directly or indirectly (e.g., directly inhibit some enzymes, bind to the membrane and/or impede the function of some phospholipases or change the pH and perturb the functioning of some proteases).

CAD molecules usually have a hydrophobic ring structure and a side chain with a cationic amine group (weak base, usually a primary, secondary or tertiary nitrogen atom N bound to a carbon C of an alkyl chain). CADs are in general not fully ionized at a physiologic pH and have an overall hydrophobicity that can be monitored using computed log P values. The amine group (N) of these compounds becomes permanently protonated in acidic compartments (e.g., endosomes or lysosomes). The drugs are then trapped and concentrated, inducing membrane structure perturbation and in some cases compromising cell viability. We defined cationic compounds as molecule with a basic pKa value above >7.1, which indicates that these substances are mainly protonated a low pH values.¹⁴ Indeed, in the endosomal-lysosomal system, the pH values are in the range of 6.5 to about 4.5. Amphiphilic compounds have, by definition, both hydrophilic and hydrophobic chemical moieties in their structures. A computed log P in the range of 2-3 to 9 combined with the presence of a positively charged N group provide an approximate definition of the amphiphilic character of a small drug-like compound. Using this reasoning, nicotine could partially fit the definition of CAD.

The log P values (c Log P) (logarithm of the octanol/water partition coefficient water/octanol system, estimation of lipophilicity, high values indicate hydrophobic molecules while low values suggest that the molecule is hydrophilic) were computed with DataWarrior using the unionized (neutral) form of the molecule as input.¹⁵ pKa values to estimate the protonation state of the most basic group were computed with the ChemAxon chemistry toolkit (https://chemaxon.com/). The number of basic nitrogen(s) was estimated at physiological pH and at pH=6 (i.e., as in the endosome). Numerous compounds should be protonated at pH=6 and indeed chloroquine and hydroxychloroquine are known to have two charged amine groups at this pH. At pH=7.4 however, many of these compounds should still be partially positively charged. Hundreds of molecules are known to induce phospholipidosis in vitro. We gathered this information from the literature.^{14, 16, 17} When the experimental data were missing, the property was predicted with our FAF-Drugs4 web server.¹⁸ In the table, “y” means Yes and “n” means No.

TABLE 2
Additional drugs with predicted antiviral activities identified by clustering
molecules commonly given to the psychiatric patients in our Department
(Table 1) with 4642 other approved drugs. The metrics used to evaluate
chemical similarity involved the merging of a circular fingerprint algorithm
with a 3D-pharmacophore approach. Visualization of the molecules to
select the compounds involved the use of T-distributed Stochastic Neighbor
Embedding and the creation of 2D similarity network maps. 17 extra
molecules were identified and considered relevant for the treatment and/or
prevention of Covid-19. These compounds are highly similar to the
molecules used in the Henri Mondor psychiatric department. Also, these
17 extra molecules belong to the CAD (cationic amphiphilic drugs)
category but Loratadine. This compound is not expected to carry a
positive charge at pH = 7, but will carry a positive charge after
metabolism in the human body. For instance, Desloratadine, the active
metabolite of Loratadine, is predicted to display such positive charge.

| | Class | CAD |
|---|---|---|
| Loratadine | antihistamine | Partial, becomes CAD after |
| | | metabolism in the human body |
| Desloratadine | antihistamine | Yes |
| Rupatadine | antihistamine | Yes |
| Azatadine | antihistamine | Yes |
| Oxomemazine | antihistamine | Yes |
| Promazine | antipsychotic | Yes |
| Profenamine | antihistamine | Yes |
| Methdilazine | antihistamine | Yes |
| Perazine | antipsychotic | Yes |
| Perphenazine | antipsychotic | Yes |
| Pizotifen | antihistamine | Yes |
| Ketotifen | antihistamine | Yes |
| Cyproheptadine | antihistamine | Yes |
| Diphenhydramine | antihistamine | Yes |
| Orphenadrine | antihistamine | Yes |
| Bromodiphenhydramine | antihistamine | Yes |
| Azelastine | antihistamine | Yes |

TABLE 3
Half-maximal inhibitory concentrations (i.e. IC50) of
Desloratadine, Azelastine or Promethazine.

| | IC50 (μM) |
|---|---|
| Desloratadine | 5.75 |
| Azelastine | 5.75 |
| Promethazine | 9.22 |

### REFERENCES

Throughout this application, various references describe the state of the art to which this invention pertains. The disclosures of these references are hereby incorporated by reference into the present disclosure.
1. Farha M A, Brown E D. Drug repurposing for antimicrobial discovery. Nat Microbiol 2019; 4(4): 565-77.
2. Grimsey E M, Piddock L J V. Do phenothiazines possess antimicrobial and efflux inhibitory properties? FEMS Microbiol Rev 2019; 43(6): 577-90.
3. Yao Q, Wang P, Wang X, et al. Retrospective study of risk factors for severe SARS-Cov-2 infections in hospitalized adult patients. Pol Arch Intern Med 2020.
4. Vieta E, Perez V, Arango C. Psychiatry in the aftermath of COVID-19. Rev Psiquiatr Salud Ment 2020.
5. Li H J, Gao D S, Li Y T, Wang Y S, Liu H Y, Zhao J. Antiviral effect of lithium chloride on porcine epidemic diarrhea virus in vitro. Res Vet Sci 2018; 118: 288-94.
6. Melander R J, Melander C. The Challenge of Overcoming Antibiotic Resistance: An Adjuvant Approach? ACS Infect Dis 2017; 3(8): 559-63.
7. Bilinska K, Jakubowska P, C S VONB, Butowt R. Expression of the SARS-CoV-2 Entry Proteins, ACE2 and TMPRSS2, in Cells of the Olfactory Epithelium: Identification of Cell Types and Trends with Age. ACS Chem Neurosci 2020.
8. Shang J, Wan Y, Luo C, et al. Cell entry mechanisms of SARS-CoV-2. Proc Natl Acad Sci USA 2020.
9. Salata C, Calistri A, Parolin C, Baritussio A, Palu G. Antiviral activity of cationic amphiphilic drugs. Expert Rev Anti Infect Ther 2017; 15(5): 483-92.
10. Ekins S, Mottin M, Ramos P, et al. Deja vu: Stimulating open drug discovery for SARS-CoV-2. Drug Discov Today 2020.
11. Johansen L M, DeWald L E, Shoemaker C J, et al. A screen of approved drugs and molecular probes identifies therapeutics with anti-Ebola virus activity. Sci Transl Med 2015; 7(290): 290ra89.
12. Dyall J, Coleman C M, Hart B J, et al. Repurposing of clinically developed drugs for treatment of Middle East respiratory syndrome coronavirus infection. Antimicrob Agents Chemother 2014; 58(8): 4885-93.
13. Gordon D E, Jang G M, Bouhaddou M, et al. A SARS-CoV-2 protein interaction map reveals targets for drug repurposing. Nature 2020.
14. Muehlbacher M, Tripal P, Roas F, Kornhuber J. Identification of drugs inducing phospholipidosis by novel in vitro data. Chem Med Chem 2012; 7(11): 1925-34.
15. Sander T, Freyss J, von Korff M, Rufener C. DataWarrior: an open-source program for chemistry aware data visualization and analysis. J Chem Inf Model 2015; 55(2): 460-73.
16. Breiden B, Sandhoff K. Emerging mechanisms of drug-induced phospholipidosis. Biol Chem 2019; 401(1): 31-46.
17. Przybylak K R, Alzahrani A R, Cronin M T. How does the quality of phospholipidosis data influence the predictivity of structural alerts? J Chem Inf Model 2014; 54(8): 2224-32.
18. Lagorce D, Bouslama L, Becot J, Miteva M A, Villoutreix B O. FAF-Drugs4: free ADME-tox filtering computations for chemical biology and early stages drug discovery. Bioinformatics 2017; 33(22): 3658-60.
19. Cao Y, Charisi A, Cheng L C, Jiang T, Girke T. ChemmineR: a compound mining framework for R. Bioinformatics 2008; 24(15): 1733-4.
20. Hoffmann M, Kleine-Weber H, Schroeder S, et al. SARS-CoV-2 Cell Entry Depends on ACE2 and TMPRSS2 and Is Blocked by a Clinically Proven Protease Inhibitor. Cell 2020; 181(2): 271-80 e8.
21. Letko M, Marzi A, Munster V. Functional assessment of cell entry and receptor usage for SARS-CoV-2 and other lineage B betacoronaviruses. Nat Microbiol 2020; 5(4): 562-9.
22. Ou X, Liu Y, Lei X, et al. Characterization of spike glycoprotein of SARS-CoV-2 on virus entry and its immune cross-reactivity with SARS-CoV. Nat Commun 2020; 11(1): 1620.
23. Millet J K, Whittaker G R. Physiological and molecular triggers for SARS-CoV membrane fusion and entry into host cells. Virology 2018; 517: 3-8.
24. Yang N, Shen H M. Targeting the Endocytic Pathway and Autophagy Process as a Novel Therapeutic Strategy in COVID-19. Int J Biol Sci 2020; 16(10): 1724-31.
25. Wang H, Yang P, Liu K, et al. SARS coronavirus entry into host cells through a novel clathrin- and caveolae-independent endocytic pathway. Cell Res 2008; 18(2): 290-301.
26. Inoue Y, Tanaka N, Tanaka Y, et al. Clathrin-dependent entry of severe acute respiratory syndrome coronavirus into target cells expressing ACE2 with the cytoplasmic tail deleted. J Virol 2007; 81(16): 8722-9.
27. Dyall J, Nelson E A, DeWald L E, et al. Identification of Combinations of Approved Drugs With Synergistic Activity Against Ebola Virus in Cell Cultures. J Infect Dis 2018; 218(suppl_5): S672-S8.
28. Fan H, Du X, Zhang J, et al. Selective inhibition of Ebola entry with selective estrogen receptor modulators by disrupting the endolysosomal calcium. Sci Rep 2017; 7: 41226.

1. A method of treating a coronavirus infection in a subject in need thereof comprising administering to the patient a therapeutically effective amount of at least one drug selected from the group consisting of Desloratadine, Promethazine, Loratadine, Azelastine, Pizotifen, Bromodiphenhydramine, Diphenhydramine, Cyproheptadine, Oxomemazine, Cetirizine, Hydroxyzine, Alimemazine, Amisulpride, Aripiprazole, Citalopram, Clozapine, Cyamemazine, Diazepam, Escitalopram, Lorazepam, Melatonin, Quetiapine, Sertraline, Valproate, Zopiclone, Rupatadine, Azatadine, Promazine, Profenamine, Methdilazine, Perazine, Perphenazine, Ketotifen, Orphenadrine and Lithium.

2. The method of claim 1 wherein the at least one drug is selected from the group consisting of Desloratadine, Promethazine, Loratadine and Azelastine.

3. The method of claim 1 wherein the at least one drug is selected from the group consisting of Desloratadine, Promethazine and Loratadine.

4. The method of claim 1 wherein the at least one drug is selected from the group consisting of Desloratadine and Loratadine.

5. The method of claim 1 wherein the at least one drug is Desloratadine.

6. The method of claim 1 wherein at least two drugs are administered to the patient.

7. The method of claim 1 wherein the at least one drug is administered to the subject in combination with at least one other therapeutic agent;

8. The method of claim 1 wherein the at least one drug is administered in combination with at least one antibody.

9. The method of claim 8 wherein the at least one antibody is bamlanivimab, casirivimab, cilgavimab, regdanvimab, sotrovimab, tixagevimab, etesevimab, imdevimab, tocilizumab, COR101, MR17, SR4, COV2-2064, VHH-72, TY-027 or JMB2002.

10. The method of claim 1 wherein the at least one drug is administered in combination with at least one vaccine.

11. The method of claim 10 wherein the at least one vaccine is mRNA-1273, BNT162b2, AZD1222, CVnCoV vaccine, NVX-CoV2373, BBIBP-CorV, Ad26.COV2.S, Ad5-nCov, Gam-COVID-Vac, CoVLP, BBV152-COVAXIN, AG0302-COVID19, SCB-2019, QazCovid-in, UB-612 or EpiVacCorona.

12. The method of claim 1 wherein the at least one drug is administered to the subject in combination with Azithromycin.

13. The method of claim 1 wherein the at least one drug is administered to the subject in combination with at least one drug selected from the group consisting of Chloroquine, Hydroxyl-chloroquine, Nicotine, Clemastine, Cloperastine, and Haloperidol.

14. The method of claim 7, wherein the at least one other therapeutic agent is at least one other antiviral agent.

15. The method of claim 14, wherein the at least one other antiviral agent is selected from the group consisting of remdesivir, lopinavir, and ritonavir.
